(19) United States
(12) Patent Application Publication
(10) Pub. No.: US 2012/0203101 A1
(43) Pub. Date: Aug. 9, 2012

(54) METHODS FOR OPTOACOUSTIC GUIDANCE AND CONFIRMATION OF PLACEMENT OF NOVEL INDWELLING MEDICAL APPARATUS

(75) Inventors: Donald S. Prough, Galveston, TX (US); Rinat O. Esenaliev, League City, TX (US); Daneshvari R. Solanki, League City, TX (US); Michael Kinsky, League City, TX (US); Yuriy Petrov, Galveston, TX (US); Irene Petrov, Galveston, TX (US)

(73) Assignee: BOARD OF REGENTS, THE UNIVERSITY OF TEXAS SYSTEM, Austin, TX (US)

(21) Appl. No.: 13/179,482

(22) Filed: Jul. 9, 2011

Related U.S. Application Data
(60) Provisional application No. 61/363,086, filed on Jul. 9, 2010.

(51) Int. Cl. A61M 16/04; A61B 6/00

(52) U.S. Cl. 600/424

(57) ABSTRACT

Indwelling medical apparatus including one optoacoustic discernible member or a plurality of optoacoustic discernible members and methods for optoacoustic guidance and confirmation of placement of optoacoustically discernible indwelling medical apparatus.

### RELATED APPLICATIONS

This application claims the benefit of and priority to U.S. Provisional Patent Application Ser. No. 61/363,086 filed Jul. 9, 2010 (Jul. 9, 2010).

### BACKGROUND OF THE INVENTION

1. Field of the Invention

Embodiments of this invention relate to indwelling medical apparatus with optoacoustic discernable member and methods for optoacoustic guidance and confirmation of placement of indwelling medical apparatus.

More particularly, embodiments of this invention relate to indwelling medical apparatus with optoacoustic discernable member and methods for optoacoustic guidance and confirmation of placement of indwelling medical apparatus, where the apparatus includes one optoacoustic discernible member or a plurality of optoacoustic discernible members.

2. Description of the Related Art

Improper placement or positioning of an endotracheal tube may be lethal. Correct placement and positioning of an endotracheal tube is an essential component of life support during resuscitation from cardiac arrest, during stabilization and surgery after severe multiple trauma, during critical illnesses requiring airway and ventilatory support, during most surgical procedures under general anesthesia and during postoperative mechanical ventilatory support. To function properly in ventilating the lungs, an endotracheal tube must be inserted into the trachea, must be properly positioned in the mid-trachea and must remain properly positioned until the endotracheal tube is no longer necessary. However, endotracheal tubes are often misplaced, particularly when placed in emergency circumstances, and endotracheal tube misplacement contributes to morbidity and mortality. Katz and Falk (Katz S H, Falk J L: “Misplaced Endotracheal Tubes by Paramedics in an Urban Emergency Medical Services System.” Ann. Emerg Med. 2001; 37: 32-7) reported on a series over an eight-month interval of 108 patients who were intubated by emergency medicine personnel before arrival at a single-hospital Emergency Department (ED). On arrival at the ED, 25% (27/108) of endotracheal tubes were misplaced. Eighteen of 27 were in the esophagus; of those 18 patients, 56% died in the ED. In nine of the 27 patients, the endotracheal tube was too deep (below the carina) or remained in the hypopharynx above the vocal cords; of those patients, 33% died in the ED. Li (Li J: “Capnography Alone Is Imperfect for Endotracheal Tube Placement Confirmation During Emergency Intubation.” J Emerg Med. 2001; 20: 223-9) reported data, provided by the National Emergency Airway Registry database, regarding emergency endotracheal intubation performed in 24 participating hospital EDs from August 1997 to September 1999. Of 4,602 attempted emergency endotracheal intubations, the number of inadvertent esophageal intubations was 180, representing 4% of emergency intubations. Of these, ten (6% of all esophageal intubations) were initially unrecognized.

Misplacement of an endotracheal tube contributes to morbidity and mortality in several ways. Placement in the esophagus rather than in the trachea results in failure to effectively provide oxygen and remove carbon dioxide. Even a single breath administered while a tube is improperly positioned in the esophagus risks gastric inflation and promotes regurgitation and aspiration of gastric contents. Positioning of an endotracheal tube insufficiently far into the trachea risks laryngeal damage from cuff pressure on the structures in the larynx and, of greater immediate concern, risks accidental withdrawal into the pharynx. Positioning of an endotracheal tube too deeply may result in intubation of a main-stem bronchus, usually the right, causing hypoxemia because of failure to ventilate the opposite, usually the left, lung. Even a properly positioned endotracheal tube may subsequently move during taping (used to secure the endotracheal tube), retaping or changes in patient position. Displacement after apparently appropriate placement most commonly occurs in obese patients, females, children and patients undergoing laparoscopy or placement in the Trendelenburg (head-down) position (Weiss M, Dullenkopf A, Bottcher S, Schmitz A, Stutz K, Gysin C, Gerber A C: “Clinical Evaluation of Cuff and Tube Tip Position in a Newly Designed Paediatric Preformed Oral Cuffed Tracheal Tube.”Br. J. Anaesth. 2006; 97: 695-700; Kim J H, Hong D M, Oh A Y, Han S H: Tracheal shortening during laparoscopic gynecologic surgery. Acta Anaesthesiol. Scand. 2007; 51: 235-8; Weiss M, Gerber A C, Dullenkopf A: Appropriate placement of intubation depth marks in a new cuffed paediatric tracheal tube. Br. J. Anaesth. 2005; 94: 80-7; Ezri T, Hazin V, Warters D, Szmuk P, Weinbroum A A: “The Endotracheal Tube Moves More Often in Obese Patients Undergoing Laparoscopy Compared with Open Abdominal Surgery.” Anesthesia and Analgesia 2003; 96: 278-82, table; Harris E A, Arheart K L, “Penning Dh: Endotracheal Tube Malposition Within the Pediatric Population: a Common Event Despite Clinical Evidence of Correct Placement.” Can. J. Anaesth. 2008; 55: 685-90; and Weiss M, Knirsch W, Kretschmar O, Dullenkopf A, Tomaske M, Balmer C, Stutz K, Gerber A C, Berger F: “Tracheal Tube-tip Displacement in Children During Head-neck Movement—a Radiological Assessment.” Br. J Anaesth. 2006; 96: 486-91). Because misplacement of an endotracheal tube can be lethal, proper positioning must be confirmed immediately after initial placement and must subsequently be monitored so that later tube displacement can promptly be recognized and corrected.

Proper insertion and positioning of endotracheal tubes customarily is performed or supervised by the most expert individual available, but expertise in endotracheal tube placement and maintenance varies widely by training and location. For respiratory support during surgery, placement is usually performed by anesthesiologists or nurse anesthetists, who typically are highly experienced and intubate patients on a daily basis.

Moreover, most elective surgical patients have relatively good physiological reserves, risk of aspiration is lessened due to precautions and are intubated in highly controlled, nonemergent circumstances.

In hospitalized patients outside the surgery suite, endotracheal tube placement is usually performed as an emergency life-support procedure by a variety of physicians and nonphysicians, depending on the size and complexity of a hospital. Patients requiring emergency intubation usually have severe physiologic compromise, such as respiratory failure and cardiac arrest, and often must be intubated under poorly controlled circumstances by personnel with highly variable experience and expertise. These patients are particularly vulnerable to episodic hypoxemia. In EDs, placement is usually performed by emergency physicians, some of whom have considerable training, experience and expertise. However, some do not. In smaller hospitals during night shifts and on weekends, endotracheal tube placement is often performed by respiratory therapists, whose training varies widely and who may rarely have the opportunity to practice intubation.

In out-of-hospital situations, placement is usually performed by emergency medicine technicians or paramedics, whose experience and training often are limited. The inevitable disparities in experience and expertise between ED physicians, respiratory therapists, anesthesiologists and out-of-hospital emergency responders are compounded in emergency circumstances by less-than-optimal conditions and limited monitoring equipment. These important factors further reduce the chances of proper initial placement and subsequent maintenance of proper positioning of endotracheal tubes.

After endotracheal tube placement before surgery, patients subsequently remain in a highly monitored, stable environment, in which endotracheal tube position can be constantly monitored by an anesthesiologist or nurse anesthetist who can recognize tube displacement and intervene. Patients who are endotracheally intubated outside surgical suites or outside hospitals typically must be transported to other locations for definitive therapy, diagnostic imaging or intensive care. In each environment and during transport, because misplacement of an endotracheal tube can be lethal, proper positioning must be confirmed immediately after initial placement and must subsequently be monitored so that later tube displacement can promptly be recognized and corrected. Currently available technology is unsuitable for monitoring of endotracheal position, especially by personnel of limited experience.

The current gold standards of clinical practice for confirmation of endotracheal tube position include: (1) Direct visualization of the endotracheal tube entering the trachea, (2) Auscultation to confirm bilateral, symmetrical breath sounds and absence of air entry over the epigastrium (to exclude esophageal intubation) and (3) Detection of exhaled carbon dioxide to confirm placement in the lungs.

Salem (Salem M R: “Verification of Endotracheal Tube Position.” Anesthesiol. Clin. North America. 2001; 19: 813-39) has nicely summarized the pitfalls of each of these techniques—although each is relatively reliable, each also is associated with errors, the consequences of which can be grave. In some patients, visualization of the larynx is inadequate to confirm endotracheal tube placement. Auscultation is prone to both false-positive and false-negative findings. The dramatic decrease in respiratory complications of anesthesia during the past thirty years is certainly attributable in part to expeditious recognition and correction of esophageal intubation (Salem M R: “Verification of Endotracheal Tube Position.” Anesthesiol. Clin. North America. 2001; 19: 813-39), although anesthesia personnel continue to be challenged by difficulty in confirming endotracheal tube placement in the mid-trachea, especially in circumstances in which post-intubation movement of a patient can result in movement of the tube within the trachea (Kim J H, Hong D M, Oh A Y, Han S H: “Tracheal Shortening During Laparoscopic Gynecologic Surgery.” Acta Anaesthesiol. Scand. 2007; 51: 235-8; Weiss M, Gerber A C, Dullenkopf A: “Appropriate Placement of Intubation Depth Marks in a New Cuffed Paediatric Tracheal Tube.” Br. J. Anaesth. 2005; 94: 80-7; Ezri T, Hazin V, Warters D, Szmuk P, Weinbroum A A: “The Endotracheal Tube Moves More Often in Obese Patients Undergoing Laparoscopy Compared with Open Abdominal Surgery.” Anesthesia and Analgesia 2003; 96: 278-82, table; Harris E A, Arheart K L, Penning D H: Endotracheal tube malposition within the pediatric population: a common event despite clinical evidence of correct placement. Can. J Anaesth. 2008; 55: 685-90; Weiss M, Knirsch W, Kretschmar O, Dullenkopf A, Tomaske M, Balmer C, Stutz K, Gerber A C, Berger F: “Tracheal Tube-tip Displacement in Children During Head-neck Movement—a Radiological Assessment.” Br. J. Anaesth. 2006; 96: 486-91; Evron S, Weisenberg M, Harow E, Khazin V, Szmuk P, Gavish D, Ezri T: “Proper Insertion Depth of Endotracheal Tubes in Adults by Topographic Landmarks Measurements.” J. Clin. Anesth. 2007; 19: 15-9; Cherng C H, Wong C S, Hsu C H, Ho S T:” Airway Length in Adults: Estimation of the Optimal Endotracheal Tube Length for Orotracheal Intubation.” J. Clin. Anesth. 2002; 14: 271-4; Sugiyama K, Yokoyama K, Satoh K, Nishihara M, Yoshitomi T: Does the Murphy eye reduce the reliability of chest auscultation in detecting endobronchial intubation? Anesthesia and Analgesia 1999; 88: 1380-3). The challenges of recognizing esophageal intubation and endotracheal tube movement are much greater in emergency circumstances outside the operating room (Katz S H, Falk J L: “Misplaced Endotracheal Tubes by Paramedics in an Urban Emergency Medical Services System.” Ann. Emerg Med. 2001; 37: 32-7 and Li J: “Capnography Alone Is Imperfect for Endotracheal Tube Placement Confirmation During Emergency Intubation.” J Emerg Med. 2001; 20: 223-9). Detection of exhaled carbon dioxide by capnography functions well in physiologically stable patients during surgical anesthesia. However, in emergency circumstances, especially during cardiac arrest, capnography is less reliable because carbon dioxide exhalation is highly variable and requires ventilation. Li (Li J: “Capnography Alone Is Imperfect for Endotracheal Tube Placement Confirmation During Emergency Intubation.” J Emerg Med. 2001; 20: 223-9) quantified the sensitivity and specificity of capnography when used in emergency circumstances. Based on a meta-analysis of capnography trials that included 2,192 intubations, the sensitivity for confirmation of endotracheal intubation was 93% (95% confidence interval 92-94%) and the specificity was 97% (CI 93-99%). Therefore, for emergency intubations, the false-negative failure rate (tube in trachea but capnography indicates esophagus) was 7% and the false-positive rate (tube in esophagus but capnography indicates trachea) was 3%.

To address the clinical problems of promptly recognizing initial endotracheal tube misplacement or subsequent endotracheal tube displacement, a variety of technological aids have been suggested or developed to supplement or replace auscultation and quantitative capnography (O'Connor C J, Mansy H, Balk R A, Tuman K J, Sandler R H: “Identification of Endotracheal Tube Malpositions Using Computerized Analysis of Breath Sounds via Electronic Stethoscopes.” Anesthesia and Analgesia 2005; 101: 735-9; Cardoso M M, Banner M J, Melker R J, Bjoraker D G: “Portable Devices Used to Detect Endotracheal Intubation During Emergency Situations: a Review.” Crit Care Med. 1998; 26: 957-64; Ezri T, Khazin V, Szmuk P, Medalion B, Shechter P, Priel I, Loberboim M, Weinbroum A A: “Use of the Rapiscope vs Chest Auscultation for Detection of Accidental Bronchial Intubation in Non-obese Patients Undergoing Laparoscopic Cholecystectomy.” J. Clin. Anesth. 2006; 18: 118-23; Reicher J, Reicher D, Reicher M: “Use of Radio Frequency Identification (Rfid) Tags in Bedside Monitoring of Endotracheal Tube Position.” J. Clin. Monit. Comput. 2007; 21: 155-8; 17 Werner S L, Smith C E, Goldstein J R, Jones R A, Cydulka R K: “Pilot Study to Evaluate the Accuracy of Ultrasonography in Confirming Endotracheal Tube Placement.” Ann. Emerg Med. 2007; 49: 75-80; Li J: “A Prospective Multicenter Trial Testing the Scoti Device for Confirmation of Endotracheal Tube Placement.” J Emerg Med. 2001; 20: 231-9; and Milling T J, Jones M, Khan T, Tad-y D, Melniker L A, Bove J, Yarmush J, SchianodiCola J: “Transtracheal 2-d Ultrasound for Identification of Esophageal Intubation.” J Emerg Med. 2007; 32: 409-14). The principles of operation of the devices vary. Some qualitatively detect exhaled carbon dioxide, some utilize transmission of light from within the trachea to the skin surface, some depend on aspiration of air from the trachea, and some are based on ultrasonography. The Sonomatic Confirmation of Tracheal Intubation (SCOTI) device connects to the end of the endotracheal tube and assesses the air content of the structure within which the endotracheal tube is located, i.e., within the rigid, air-filled trachea or the flaccid esophagus (Li J: “A Prospective Multicenter Trial Testing the SCOTI Device for Confirmation of Endotracheal Tube Placement.” J Emerg Med. 2001; 20: 231-9). Fiberoptic bronchoscopy is a skill that has been used to confirm proper placement within the trachea. Chest radiographs are used in hospitalized patients to confirm proper placement at a single moment in time. Although all approaches offer advantages and provide feedback that can be helpful, no single device is sufficiently reliable to be considered the standard of care and some, such as fiberoptic bronchoscopy, require substantial skill and training.

Thus, there is a real need in the art for an easy method for monitoring and confirming proper placement of indwelling medical apparatus in an animal body, a mammalian body or a human body.

### SUMMARY OF THE INVENTION

Embodiments of the present invention provide indwelling medical apparatus including one optoacoustically discernible member or a plurality of optoacoustically discernible members, where the members absorb electromagnetic radiation (light) and generate spatially resolved pressure signals in response to the absorbed electromagnetic radiation. In certain embodiments, the electromagnetic radiation comprises near-infrared light, the optical component of the optoacoustic technique, and pressure signal comprises an ultrasound signal, the acoustic component of the optoacoustic technique.

Embodiments of the present invention provide methods for placing and monitoring the placement of indwelling medical apparatus. The methods include providing an indwelling medical apparatus including one optoacoustically discernible member or a plurality of optoacoustically discernible members, where the members absorb electromagnetic radiation (light) and generate spatially resolved pressure signals in response to the absorbed electromagnetic radiation. In certain embodiments, the electromagnetic radiation comprises near-infrared light, the optical component of the optoacoustic technique, and pressure signal comprises an ultrasound signal, the acoustic component of the optoacoustic technique. The methods also include inserting the apparatus into a body of an animal, a mammal or a human and monitoring the insertion via an optoacoustic probe situated on an external portion or an internal portion of the body proximate the apparatus as it is being inserted. The methods also include confirming placement of the apparatus through the optoacoustic monitoring. The methods may optionally include continuous, periodic, and/or intermittent monitoring of the indwelling apparatus to insure continued correct apparatus placement. In certain embodiment, the apparatus is an endotracheal tube including one optoacoustically discernible member or a plurality of optoacoustically discernible members, where the members absorb electromagnetic radiation (light) and generate spatially resolved pressure signals in response to the absorbed electromagnetic radiation, where the endotracheal tube is properly positioned in a mid-trachea and not accidentally positioned in the esophagus due to the optoacoustic monitoring of the members on the apparatus. In certain embodiment, the apparatus is an endotracheal tube having a cuff including one optoacoustically discernible member or a plurality of optoacoustically discernible members, where the members absorb electromagnetic radiation (light) and generate spatially resolved pressure signals in response to the absorbed electromagnetic radiation, where the cuff or the endotracheal tube is properly positioned in a mid-trachea and not accidentally positioned in the esophagus due to the optoacoustic monitoring of the members on the apparatus. In other embodiments, the tube and the cuff may include one or a plurality of optoacoustically discernible members.

### BRIEF DESCRIPTION OF THE DRAWINGS

The invention can be better understood with reference to the following detailed description together with the appended illustrative drawings in which like elements are numbered the same:

FIG. 1 depicts linear dependence of optoacoustic signal recorded in vivo from an endogenous pigment (blood hemoglobin) circulating in the radial artery. The depth of the radial artery beneath the skin is comparable to the depth of the trachea and the diameter of the radial artery represents a practical width for application of pigment to an endotracheal tube cuff.

FIG. 2 depicts linear dependence on concentration of effective attenuation coefficient of an exogenous dye (ICG) that was measured by analyzing optoacoustic signals recorded from ICG.

FIG. 3 depicts typical pattern recorded through a 3-mm turbid tissue phantom from 3 pigmented mm-sized optoacoustically discernible members. Arrows indicate position and diameters of the optoacoustically discernible members.

FIG. 4A depicts an endotracheal tube with 3 pigmented lines. The central line is wider than the side lines.

FIG. 4B depicts typical signals recorded from the central line (black) and one of the side lines (gray).

FIGS. 5A-H depict various embodiments of an indwelling medical apparatus or an insertable medical apparatus including one optoacoustically discernible member (A&B), two optoacoustically discernible members (C&D), three optoacoustically discernible members (E&F), and patterns of three or more optoacoustically discernible members (G&H).

FIGS. 6A-H depict various embodiments of an endotracheal apparatus including one optoacoustically discernible member, a plurality of optoacoustically discernible members or patterns of optoacoustically discernible members.

### DETAILED DESCRIPTION OF THE INVENTION

The inventors have found that an optoacoustic method can be implemented for confirming and monitoring of correct placement of indwelling medical apparatus. In certain embodiments of this optoacoustic methodology, confirming and monitoring is directed to a correct placement of endotracheal tubes in children and adults as an example of the general use of this optoacoustic methodology for confirming and monitoring insertion and correct placement of indwelling medical apparatus.

Embodiments of the present invention broadly relate to indwelling medical apparatus including one optoacoustically discernible member or a plurality of optoacoustically discernible members, where the members absorb electromagnetic radiation (light) and generate spatially resolved pressure signals in response to the absorbed electromagnetic radiation. In certain embodiments, the electromagnetic radiation comprises near-infrared light, the optical component of the optoacoustic technique, and pressure signals comprise ultrasound signals, the acoustic component of the optoacoustic technique.

Embodiments of the present invention broadly relate to methods for inserting, placing and monitoring the placement of indwelling medical apparatus. The methods include providing an indwelling medical apparatus including one optoacoustically discernible member or a plurality of optoacoustically discernible members, where the members absorb electromagnetic radiation (light) and generate spatially resolved pressure signals in response to the absorbed electromagnetic radiation. In certain embodiments, the electromagnetic radiation comprises near-infrared light, the optical component of the optoacoustic technique, and pressure signals comprise ultrasound signals, the acoustic component of the optoacoustic technique. The methods also include inserting the apparatus into a body of an animal, a mammal or a human and monitoring the insertion via an optoacoustic probe situated proximate to the apparatus as it is being inserted. The probe may be placed on an external portion of the body or an internal portion of the body, provided that the probe is proximate the apparatus or in a straight line of travel for the pressure signals propagating through the tissue of the body surrounding the apparatus during insertion. The methods also include confirming placement of the apparatus through optoacoustic monitoring. The methods may optionally include continuous, periodic, and/or intermittent monitoring of the indwelling apparatus to insure correct apparatus placement. In certain embodiments, the apparatus is an endotracheal tube including one optoacoustically discernible member or a plurality of optoacoustically discernible members, where the members absorb electromagnetic radiation (light) and generate spatially resolved pressure signals in response to the absorbed electromagnetic radiation, where the endotracheal tube is properly positioned in a mid-trachea and not accidentally positioned in the esophagus due to the optoacoustic monitoring of the members on the apparatus. In certain embodiment, the apparatus is an endotracheal tube having a cuff including one optoacoustically discernible member or a plurality of optoacoustically discernible members, where the members absorb electromagnetic radiation (light) and generate spatially resolved pressure signals in response to the absorbed electromagnetic radiation, where the cuff or the endotracheal tube is properly positioned in a mid-trachea and not accidentally positioned in the esophagus due to the optoacoustic monitoring of the members on the apparatus. In other embodiments, the tube and the cuff may include one or a plurality of optoacoustically discernible members.

Optoacoustic monitoring is used to guide and confirm placement of endotracheal tubes and to continuous or intermittent monitoring of correct placement, utilizing optoacoustically discernible members comprise a pigmented pattern that absorbs near-infrared light. When a pulsed source of near-infrared light is absorbed by the member, the member will generate a corresponding acoustic or pressure response. The frequency of the acoustic response is controlled by the wavelength and pulse duration of the pulsed light source and optical properties of the member. In certain embodiments, the pulse light produces an ultrasonic response. In certain embodiments, the pulse light produces an ultrasonic response. An optoacoustic probe is then positioned on the anterior neck to provide rapid initial assessment and subsequent intermittent, periodic, or continuous feedback regarding the positioning of the cuff of the endotracheal tube. The inventors have demonstrated that the systems and methods of this invention are capable of confirming the proper placement of the cuffs or endotracheal tubes in the mid-trachea reducing or eliminating accidental placement of the cuffs or endotracheal tubes in the esophagus.

There are several characteristics of optoacoustic technology and of the human trachea and esophagus that make optoacoustic technology ideally suited for confirming and monitoring proper placement of apparatus placed in the trachea or for placement of any other indwelling apparatus in its intended location in the body of an animal, a mammal or a human. Optoacoustic technology is based on the fact that when pulsed light (e.g., near-infrared light) encounters a chromophore or pigment, the light is absorbed producing an acoustic response. The frequency of the acoustic response depends on the wavelength, pulse duration, and optical properties of the apparatus. In embodiments involving tissues in an animal, a mammal or a human body, the wavelength and pulse duration is adjusted to induce an ultrasonic response or to generate ultrasound waves. The acoustic response (ultrasound waves) travels in a straight line from its source. The acoustic response is then received by an optoacoustic probe and provides both lateral resolution and axial resolution regarding a size and shape of the source.

Ultrasound waves propagate through tissue, but are effectively blocked by air—ultrasound waves propagate through tissue, but not air. The trachea is an air-filled cylinder that lies immediately beneath the anterior surface of the neck. When the cuff of an endotracheal tube is inflated, the cuff directly seals against an interior surface of a trachea, thereby providing a short, direct path of tissue through which the ultrasound wave is transmitted to the surface of the neck. Within a few seconds, an optoacoustic assessment will confirm that the cuff of the endotracheal tube is in correct position within the trachea (that lies between the esophagus and the anterior neck), that it is not in the esophagus in which case the wave would be stopped by the air in the trachea, i.e., reflected completely by the trachea-air interface or that it is not inserted too deeply or too shallowly in the trachea. For guidance of intubation, the pulsed near-infrared light source can be incorporated into or transmitted through a stylet, a hollow endotracheal exchange catheter, a rigid laryngoscope, a fiberoptic endoscope or incorporated into or transmitted through the endotracheal tube itself. For confirmation and monitoring of endotracheal tube position, the pulsed near-infrared light source can be incorporated into the ultrasound detection monitor (using the backward mode) or can be transmitted through a suction catheter, stylet, hollow endotracheal tube exchange catheter, a rigid laryngoscope, a fiberoptic endoscope or incorporated into or transmitted through the endotracheal tube itself (forward mode). Therefore, optoaoustic technology can either be developed as a stand-alone device or can be incorporated into and improve existing technology.

Optoacoustic guidance of endotracheal intubation and confirming and monitoring of endotracheal tube positioning has the following attributes: (1) easy to use with minimal training, (2) negligible incidence of false-positive and false-negative results, (3) nearly instantaneous feedback regarding endotracheal tube position, (4) effective confirmation of initial endotracheal tube placement at the proper cephalad/caudad orientation, (5) continuous monitoring to detect subsequent cephalad or caudad displacement, (6) no requirement for ventilation to detect endotracheal tube placement, (7) no requirement for temporary disconnection from ventilation to confirm or monitor endotracheal tube placement and (8) no requirement for patient transportation or movement to determine endotracheal tube position.

The inventors speculate that the reason that no device or methodology has become the standard of care for confirmation of placement of endotracheal tubes or other indwelling medical apparatus is that none fulfill all of the criteria that are offered by the optoacoustic methodology of this invention and that are essential for clinical and commercial success: (1) easy to use with minimal training, (2) negligible incidence of false-positive and false-negative results, (3) nearly instantaneous feedback regarding endotracheal tube position, (4) effective confirmation of initial endotracheal tube placement at a proper depth in the trachea, (5) continuous monitoring to detect subsequent cephalad or caudad displacement, (6) no requirement for ventilation to detect endotracheal tube placement, (7) no requirement for temporary disconnection from ventilation to confirm endotracheal tube placement, and (8) no requirement for patient movement to confirm endotracheal tube placement.

Direct visualization of an endotracheal tube passing the cords requires expertise in laryngoscopy, sometimes is difficult or impossible to achieve and cannot be performed continuously. Detection of exhaled carbon dioxide is particularly misleading in patients during cardiac arrest (when minimal carbon dioxide may be exhaled through the lungs) and provides a substantial incidence of false-positive and false-negative results in emergency intubations. Fiberoptic bronchoscopy requires technical expertise, interferes with ventilation and cannot be performed continuously. Chest radiography is intermittent, requires movement of a patient to perform radiography and does not provide rapid feedback. The SCOTI device requires disconnection from the ventilator, only differentiates esophageal from tracheal intubation, has an appreciable false-positive and false-negative rate (Li J: A prospective multicenter trial testing the SCOTI device for confirmation of endotracheal tube placement. J Emerg Med. 2001; 20: 231-9) and does not indicate proper position within the trachea. Ultrasound-based techniques require expertise in ultrasonography and are not suitable for continuous monitoring.

Endotracheal tube placement is a specific example of placement of a medical device or foreign body within tissues with the subsequent need to noninvasively confirm correct placement. Optoacoustic technology is ideally suited to any clinical situation in which a foreign body is placed for medical purposes, e.g., intravascular catheters, urinary bladder catheters, drainage tubes or prosthestic devices, and in which subsequent noninvasive confirmation of proper placement is required.

### DESCRIPTION OF INVENTION

The three components of the invention comprise 1) a modified indwelling medical apparatus such as an endotracheal tube, 2) a pulsed near-infrared light source and 3) an optoacoustic probe. The apparatus is modified to include one or a plurality of optoacoustically discernible member(s) attached to, affixed to, or integral with portion of the exterior surfaces of the apparatus, where the attachment or affixing may be detachable or non-detachable. For example, the cuffs of endotracheal tubes may be modified by adding a pigmented member(s) or pattern of pigmented members that absorb near-infrared light (the optical component of optoacoustic technique) and generate spatially resolved ultrasound signals (the acoustic component of optoacoustic technique). A purpose-built optoacoustic probe positioned on the anterior neck will provide rapid and, if necessary, intermittent, periodic or continuous feedback regarding the positioning of the cuff of the endotracheal tube, demonstrating that the tube is properly positioned in the trachea and is not accidentally positioned in the esophagus.

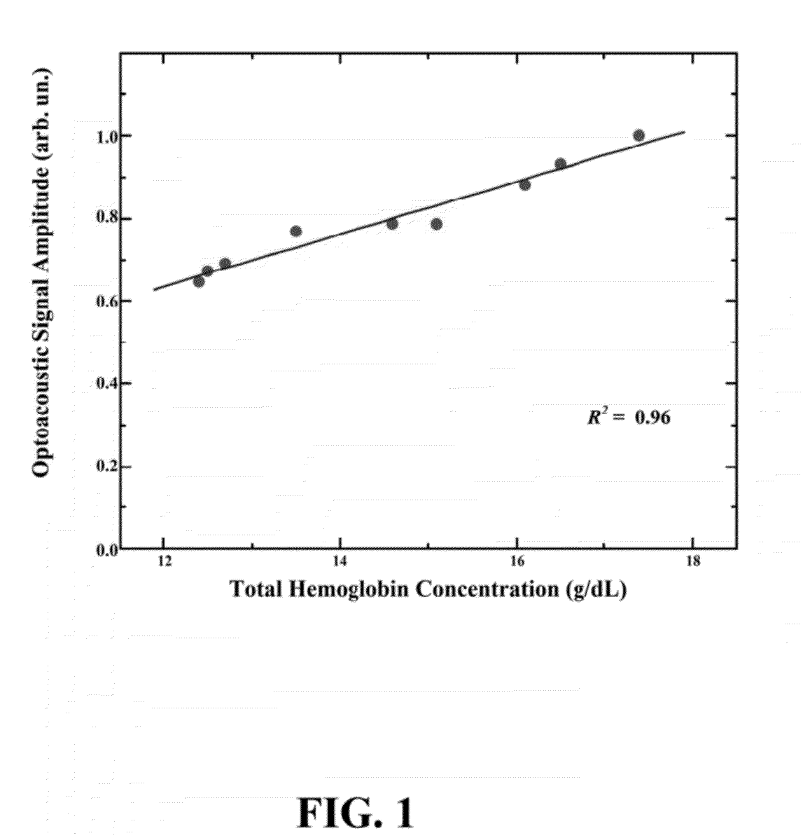

The pigmented member(s) or pattern of pigmented members may have either different absorption coefficients, sizes or shapes to provide a well-defined acoustic response. The members may also be arranged in a pattern so that the response will evidence the exact placement and potential orientation of the apparatus. The absorption coefficients of the patterns can be varied by changing concentrations of the pigments. The amplitudes and slopes of the optoacoustic signals are linearly dependent on the pigment concentrations. The inventors experimentally confirmed this in a number of in vitro and in vivo studies using exogenous and endogenous pigmented members and our optoacoustic systems in the backward mode. For instance, FIG. 1 shows the linear dependence of an optoacoustic signal recorded in vivo from an endogenous pigmented member (blood hemoglobin) circulating in the radial artery. The radial artery has a depth of 2-4 mm which is similar to the distance between the skin surface and an endotracheal tube inserted in a trachea (3-4 mm) of a patient. In addition, the diameter of the artery is about 2-3 mm that is close to the optimal width of the pigmented members lines (1.5-2 mm). Therefore, by varying the concentrations of the pigmented lines, one can create a specific pattern that can be easily recognized optoacoustically.

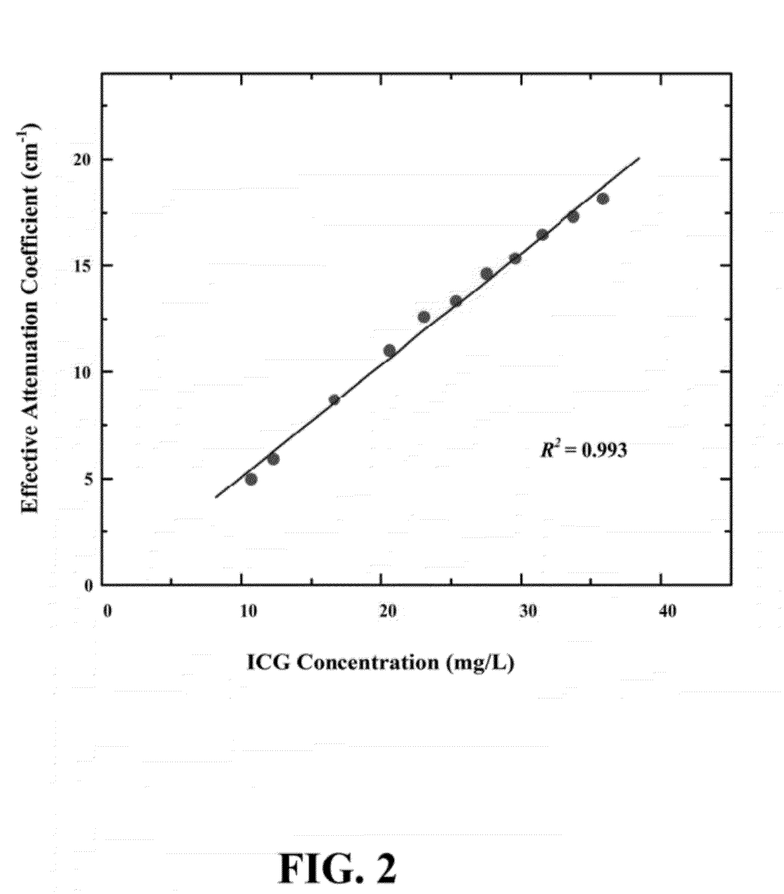

Referring now to FIG. 1, linear dependence of optoacoustic signals recorded in vivo from an endogenous pigmented member (blood hemoglobin) circulating in a radial artery. The depth of the radial artery beneath the skin is comparable to the depth of the trachea and the diameter of the radial artery represents a practical width for application of pigmented member(s) to an endotracheal tube and/or tube cuff. Referring now to FIG. 2, the linear dependence on concentration of effective attenuation coefficient of an exogenous dye, indocyanine green (ICG, which is FDA-approved for use in patients) is shown. The attenuation coefficient was measured by analyzing optoacoustic signals recorded from ICG.

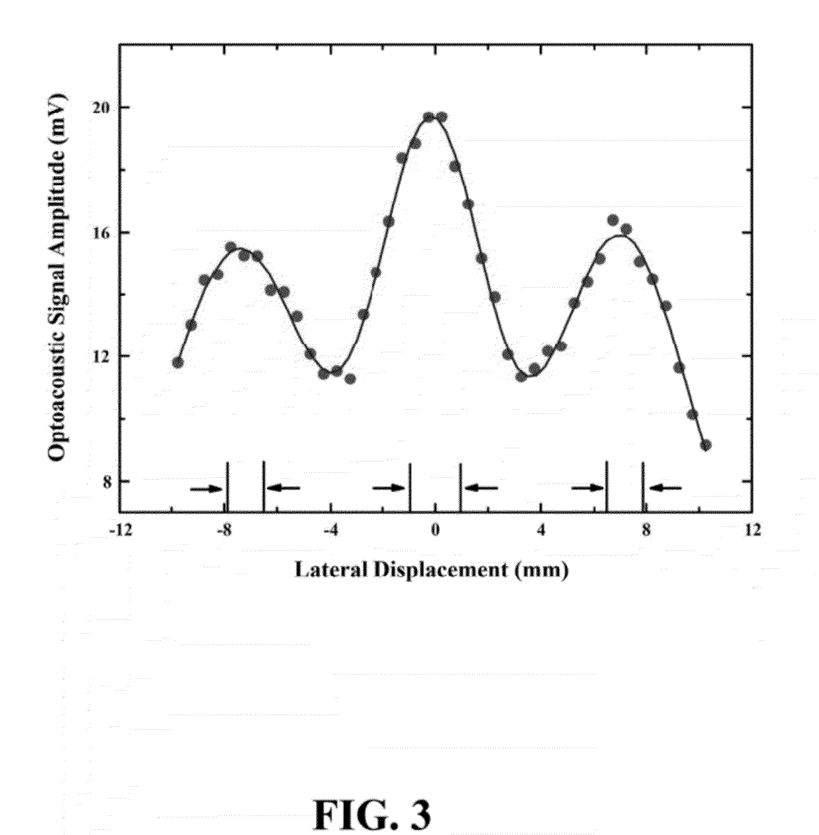

Referring now to FIG. 3, a typical pattern recorded through a 3-mm turbid tissue phantom from 3 pigmented mm-sized cylindrical members: cavities filled with hemoglobin (same concentration in all 3 cavities). The arrows indicate the positions and diameters of the cylindrical members. The central cavity had a greater diameter compared to the other two cavities. By using lateral scanning of our optoacoustic probe over the pigmented members, we recorded the pattern with 3 distinct peaks. The central peak had a high amplitude, while the side peaks had lower amplitude due to the smaller size. The optoacoustic system provided sub-millimeter lateral resolution. These data indicate that the optoacoustic system should be capable of detecting sub-millimeter displacement of pigmented objects in tissues. Therefore, the optoacoustic method should provide sub-millimeter accuracy of endotracheal tube placement and position monitoring.

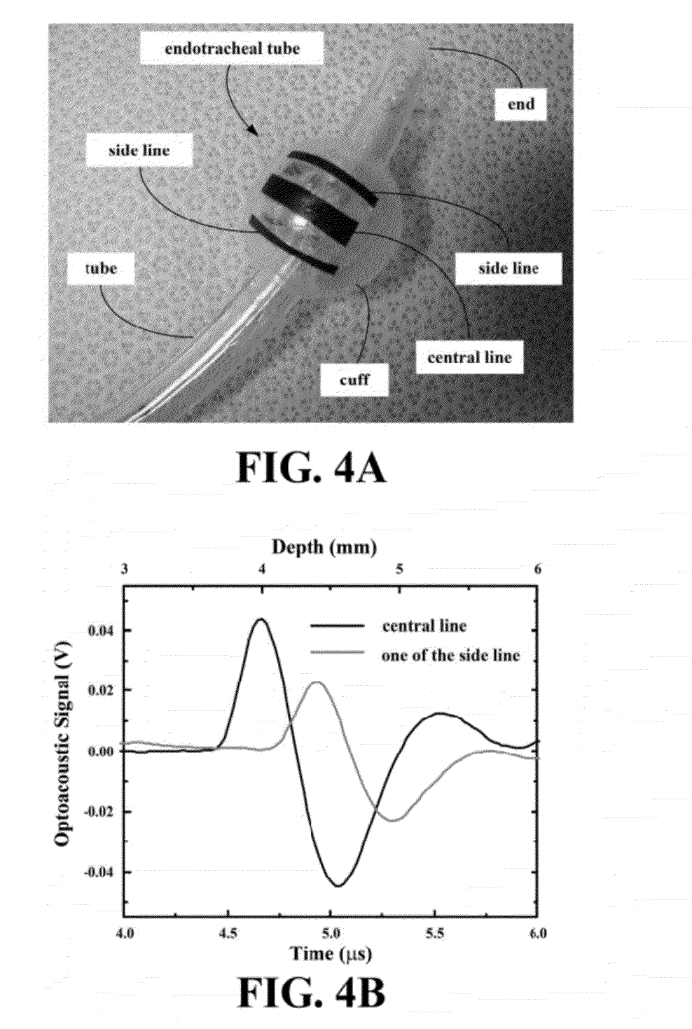

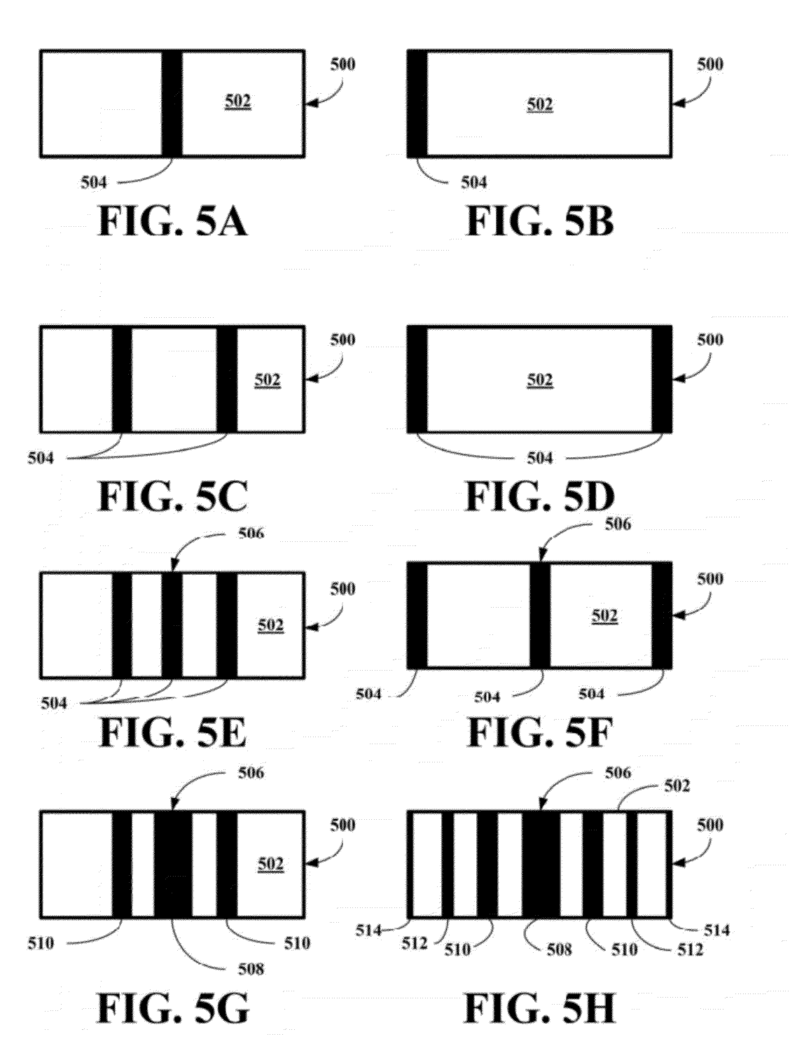

To create a prototype endotracheal tube, we used a dark, thin plastic tape to make three pigmented lines (mm-sized) on the endotracheal tube (FIG. 4A) and recorded optoacoustic signals from the lines through a 4-mm turbid tissue phantom. The central line was wider than the side lines. FIG. 4B shows typical signals recorded from the central line (black) and one of the side lines (gray). The signal from the central line was greater because the central line was wider than the side lines and was recorded approximately 0.3 microseconds earlier because the side line was 0.5 mm further from the detector due to the curvature of the cuff.

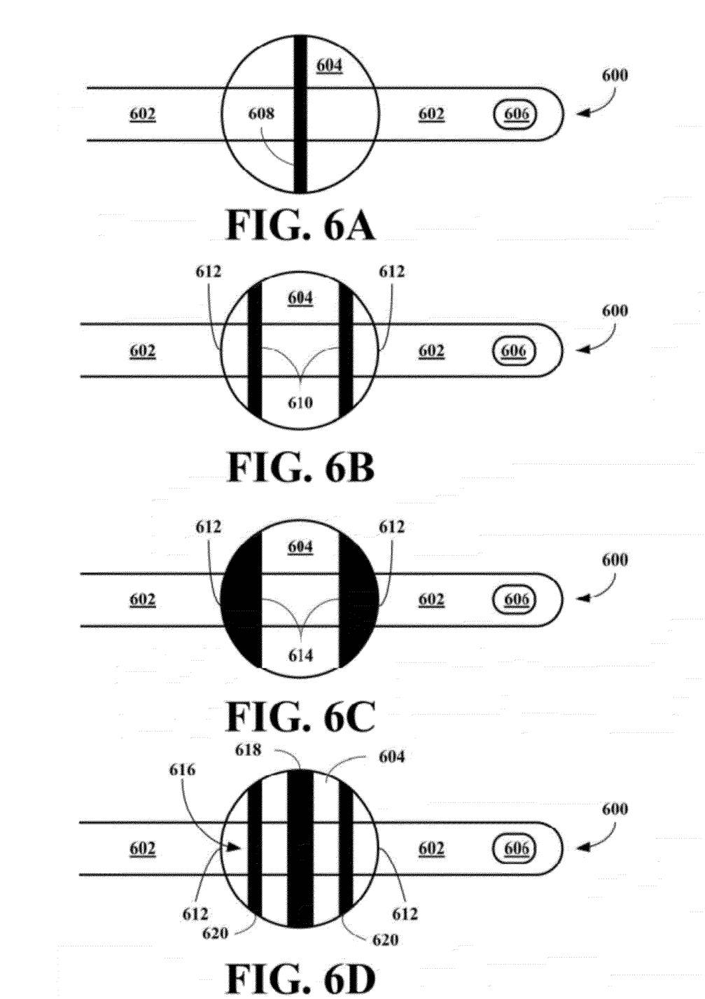

Referring now to FIGS. 5A-H, various indwelling medical apparatus are shown that include one, a plurality or a pattern of optoacoustically discernible members. Looking at FIG. 5A, the apparatus 500 is shown to include a body 502 and one centrally disposed optoacoustically discernible member 504. Looking at FIG. 5B, the apparatus 500 is shown to include a body 502 and one end disposed optoacoustically discernible member 504. Looking at FIG. 5C, the apparatus 500 is shown to include a body 502 and two centrally disposed optoacoustically discernible members 504. Looking at FIG. 5D, the apparatus 500 is shown to include a body 502 and one end disposed optoacoustically discernible members 504. Looking at FIG. 5E, the apparatus 500 is shown to include a body 502 and a pattern 506 of three centrally disposed optoacoustically discernible members 504. Looking at FIG. 5F, the apparatus 500 is shown to include a body 502 and a pattern 506 of three disposed optoacoustically discernible members 504, one centrally disposed and two end disposed. Looking at FIG. 5G, the apparatus 500 is shown to include a body 502 and a pattern 506 including a center member 508 and two side members 510, where the center member 508 is wider than the two side members 510. Looking at FIG. 5H, the apparatus 500 is shown to include a body 502 and a pattern 506 including a center member 508 and three side members 510, 512, and 514. The center member 508 is wider than the nearest side members 510. The nearest side members 510 are wider than the next two side members 512, which are wider than the end members 514. In all of the embodiments of FIG. 5, the members may encircle the apparatus 500 or may be disposed on only a portion of each side of the apparatus.

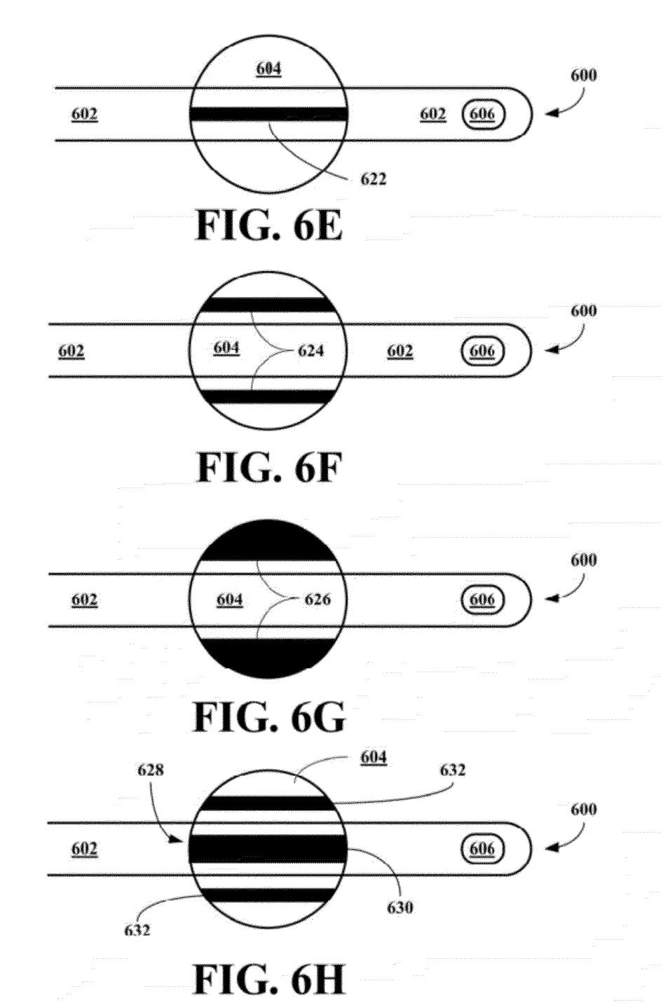

Referring now to FIGS. 6A-H, various indwelling medical apparatus are shown that including one, a plurality or a pattern of optoacoustically discernible member(s). Looking at FIG. 6A, the apparatus 600 is shown to include a tube 602 having a cuff 604 and a distal aperture 606. The cuff 604 includes one centrally and vertically disposed optoacoustically discernible member 608. Looking at FIG. 6B, the apparatus 600 is shown to include a tube 602 having a cuff 604 and a distal aperture 606. The cuff 604 includes two optoacoustically discernible members 610 disposed vertically near ends 612 of the cuff 604. Looking at FIG. 6C, the apparatus 600 is shown to include a tube 602 having a cuff 604 and a distal aperture 606. The cuff 604 includes two optoacoustically discernible vertical end members 614. Looking at FIG. 6D, the apparatus 600 is shown to include a tube 602 having a cuff 604 and a distal aperture 606. The cuff 604 includes a pattern 616 of a center wide vertical optoacoustically discernible member 618 and two side narrower optoacoustically discernible vertical members 620. Looking at FIG. 6E, the apparatus 600 is shown to include a tube 602 having a cuff 604 and a distal aperture 606. The cuff 604 includes one centrally disposed optoacoustically discernible horizontal member 622. Looking at FIG. 6F, the apparatus 600 is shown to include a tube 602 having a cuff 604 and a distal aperture 606. The cuff 604 includes two optoacoustically discernible horizontal members 624. Looking at FIG. 6G, the apparatus 600 is shown to include a tube 602 having a cuff 604 and a distal aperture 606. The cuff 604 includes two optoacoustically discernible horizontal end members 626. Looking at FIG. 6H, the apparatus 600 is shown to include a tube 602 having a cuff 604 and a distal aperture 606. The cuff 604 includes a pattern 628 of a center wide horizontal optoacoustically discernible member 630 and two side narrower optoacoustically discernible horizontal members 632. In all of the embodiments of FIG. 6, the members may be continuous bands or discontinuous bands. Additionally, for those embodiments including a plurality of members, the members may be separated by known gaps, where the gaps and widths may be designed to give rise to an optoacoustic response pattern that may be used to insure proper apparatus placement, especially if the site has a discernible response as well. Of course, the members on the cuff do not have to be continuous or vertically or horizontally oriented, but can be disposed at any angle. Additionally, the members can form crossing patterns.

Variation of both absorption coefficient and width of the lines may provide additional contrast in the patterns.

Embodiments of this invention allow confirmation that a catheter, introducer or introducer wire is located intravascularly. For that application, the optical input would come from an optical fiber incorporated within a catheter, dilator, introducer or introducer wire and the acoustic detector would be incorporated within a catheter, dilator, introducer or introducer wire. The transmission of the acoustic signal generated by oxygenated and deoxygenated hemoglobin would provide confirmation that the device was located within an artery or vein. (Currently there are intravascular optical devices that measure oxygenated and deoxygenated hemoglobin with multiwavelength optical devices—optoacoustic measurement of saturation would be a good way to confirm that a catheter was where it was intended to be.)

Embodiments of this invention allow confirmation of the positioning of a subcutaneous probe by incorporation of an absorbing pigmented member in the probe and identification of the position and depth of the probe by an external optoacoustic device.

Embodiments of this invention allow confirmation that an indwelling catheter, such as a chronic catheter placed through the antecubital vein remains in proper position by incorporation of an absorbing pigmented members in the catheter and identification of the position and depth of the catheter by an external optoacoustic device.

All references cited herein are incorporated by reference. Although the invention has been disclosed with reference to its preferred embodiments, from reading this description those of skill in the art may appreciate changes and modification that may be made which do not depart from the scope and spirit of the invention as described above and claimed hereafter.

We claim:

1. An indwelling medical apparatus comprising a body and one optoacoustically discernible member or a plurality of optoacoustically discernible members attached to, affixed to or integral with the body, where the members absorb pulsed electromagnetic radiation (light) and generate spatially resolved pressure signals in response to the absorbed pulsed electromagnetic radiation.

2. The apparatus of claim 1, wherein the electromagnetic radiation comprises near-infrared light, the optical component, and pressure signal comprises an ultrasound signal, the acoustic component.

3. The apparatus of claim 1, wherein the optoacoustically discernible members form a pattern.

4. The apparatus of claim 3, wherein the pattern comprises members of different widths spaced apart by different separations or gaps.

5. The apparatus of claim 1, wherein the apparatus comprises an insertable medical apparatus.

6. The apparatus of claim 1, wherein the apparatus comprises an indwelling medical apparatus

7. The apparatus of claim 6, wherein the indwelling medical apparatus comprises an endotracheal apparatus including a tube and a cuff.

8. The apparatus of claim 7, wherein the tube includes the members.

9. The apparatus of claim 7, wherein the cuff includes the members.

10. The apparatus of claim 7, wherein the tube and the cuff include the members.

11. The apparatus of claim 1, wherein the members are detachably attached or affixed to the body.

12. The apparatus of claim 1, wherein the members are non-detachably attached or affixed to the body.

13. The apparatus of claim 1, wherein the members are integral with the body.

14. A method, for placing and monitoring the placement of indwelling medical apparatus, comprising:
providing an indwelling medical apparatus comprising a body and one optoacoustically discernible member or a plurality of optoacoustically discernible members attached to, affixed to or integral with the body, where the members absorb pulsed electromagnetic radiation (light) and generate spatially resolved pressure signals in response to the absorbed pulsed electromagnetic radiation;
inserting the apparatus into a body of an animal, a mammal or a human and
monitoring the insertion via an optoacousitc probe situated proximate the apparatus or in proximity to the apparatus as it is being inserted.

15. The method of claim 14, further comprising:
confirming placement of the apparatus via the optoacoustic monitoring.

16. The method of claim 14, further comprising:
continuous, periodic, and/or intermittent monitoring of the indwelling apparatus to insure correct apparatus placement.

17. The method of claim 14, wherein the electromagnetic radiation comprises near-infrared light, the optical component, and pressure signal comprises an ultrasound signal, the acoustic component.

18. The method of claim 14, wherein the optoacoustically discernible members form a pattern.

19. The method of claim 18, wherein the pattern comprises members of different widths spaced apart by different separations or gaps.

20. The method of claim 14, wherein the apparatus comprises an insertable medical apparatus.

21. The method of claim 14, wherein the apparatus comprises an indwelling medical apparatus

22. The method of claim 21, wherein the indwelling medical apparatus comprises an endotracheal apparatus including a tube and a cuff.

23. The method of claim 22, wherein the cuff includes the members.

24. The method of claim 14, wherein the members are detachably attached or affixed to the body.

25. The method of claim 14, wherein the members are non-detachably attached or affixed to the body.

26. The method of claim 14, wherein the members are integral with the body.
